(19) United States
(12) Patent Application Publication
(10) Pub. No.: US 2011/0131709 A1
(43) Pub. Date: Jun. 9, 2011

(54) SAFETY SYSTEM

(76) Inventor: Fernando Caraballo, Milwaukee, WI (US)

(21) Appl. No.: 12/962,598

(22) Filed: Dec. 7, 2010

Related U.S. Application Data
(60) Provisional application No. 61/283,631, filed on Dec. 7, 2009.

(51) Int. Cl. A42B 1/24; A61F 9/00; A61F 11/12; B23P 19/04

(52) U.S. Cl. 2/422; 2/12; 128/864; 29/428

(57) ABSTRACT

One example embodiment includes a system for protecting a worker in a dangerous environment. The system includes an eye protector. The eye protector includes one or more lenses, where the one or more lenses are configured to be positioned in front of the user's eyes and configured to protect the user's eyes. The system also includes a hearing protector, wherein the hearing protector is configured to protect the user's hearing from damage.

### CROSS-REFERENCE TO RELATED APPLICATIONS

This application claims the benefit of and priority to U.S. Provisional Patent Application Ser. No. 61/283,631 filed on Dec. 7, 2009, which application is incorporated herein by reference in its entirety.

### BACKGROUND OF THE INVENTION

Many workers are exposed daily to unsafe conditions. These conditions can lead to worker injury, such as eye injuries. Some injuries are immediate whereas others produce long-term injuries, such as hearing damage. The danger is prevalent enough that there are many local, state and federal regulations which aim to protect workers in these conditions. Employers which violate the regulations can face severe penalties.

One of the most common safety regulations is the requirement that workers wear safety equipment. The equipment required depends on the hazards present in the environment. For example, a worker working with harsh of potentially deadly chemicals may be required to wear a full body suit, whereas a worker in a machine shop might only be required to wear safety glasses.

Two of the most common types of required safety equipment are safety glasses or goggles and hearing protection. However, these are also some of the easiest safety equipment to misplace or skip using completely. Often, workers will have one piece of safety equipment handy and use that piece of safety equipment, but will ignore other required safety equipment because it is inconvenient to locate. However, this short-term convenience can lead to long-term consequences, as described above.

Another common reason for a user to stop wearing safety equipment is that the equipment becomes dirty with use. For example, safety glasses may begin to gather debris which makes it hard to see through the safety glasses. The user may not be near cleaning equipment and may, therefore, forego the use of the safety glasses.

Accordingly, there is a need in the art for a system that makes locating safety equipment convenient. In addition, there is a need in the art safety equipment that is convenient to clean. Further, there is a need on the art for safety equipment which can be easily changed, as conditions change.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a system for protecting a worker in a dangerous environment. The system includes an eye protector. The eye protector includes one or more lenses, where the one or more lenses are configured to be positioned in front of the user's eyes and configured to protect the user's eyes. The system also includes a hearing protector, wherein the hearing protector is configured to protect the user's hearing from damage.

Another example embodiment includes a system for protecting a worker in a dangerous environment. The system includes an eye protector. The eye protector includes a frame, where the frame is configured to locate the eye protector in front of the user's eyes, and one or more lenses, where the one or more lenses are held within the frame in front of the user's eyes and configured to protect the user's eyes. The system also includes a hearing protector. The hearing protector includes a left ear protector and a right ear protector where the hearing protector is configured to protect the user's hearing from damage.

Another example embodiment includes a method of manufacturing a system for protecting a worker in a dangerous environment. The method includes providing an eye protector. The eye protector includes one or more lenses, where the one or more lenses are configured to be positioned in front of the user's eyes and configured to protect the user's eyes. The method also includes providing a hearing protector, wherein the hearing protector is configured to protect the user's hearing from damage.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1A illustrates a front perspective view of a system for increasing working safety;

FIG. 1B illustrates a rear view of the system for increasing working safety;

FIG. 1C illustrates a side view of the system for increasing working safety

FIG. 2 illustrates an alternative system of increasing worker safety;

FIG. 3 illustrates an alternative system of increasing worker safety; and

FIG. 4 is a flow chart illustrating a method of manufacturing a system for increasing worker safety.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

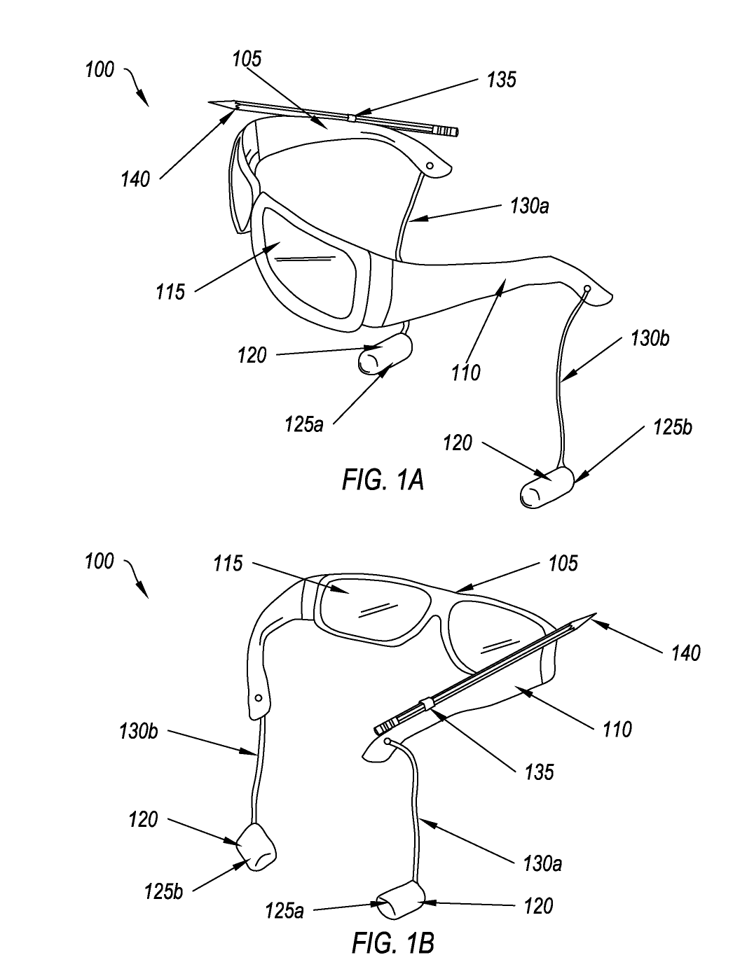

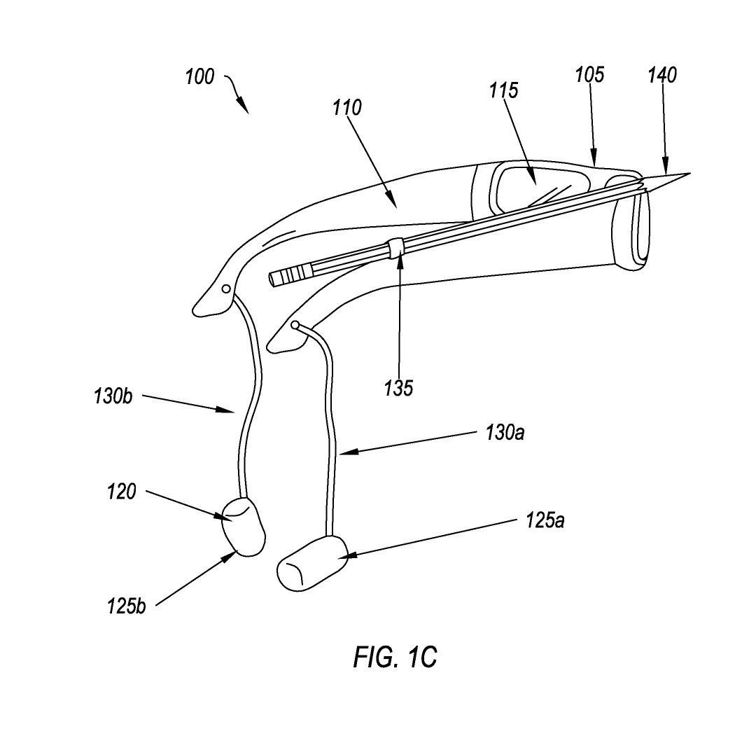

FIGS. 1A, 1B and 1C illustrate an example of a system 100 for increasing worker safety. FIG. 1A illustrates a front perspective view of the system 100 for increasing working safety; FIG. 1B illustrates a rear view of the system 100 for increasing working safety; and FIG. 1C illustrates a side view of the system 100 for increasing working safety. In at least one implementation, the system 100 can be used to protect a worker in a dangerous environment. For example, in construction zones, manufacturing areas or anywhere else that the user is likely to be exposed to loud noises or moving debris the system 100 can protect the user's eyes and hearing.

FIGS. 1A, 1B and 1C show what the system 100 can include an eye protector 105. In at least one implementation, the eye protector 105 can serve to protect the user's eyes. For example, the eye protector 105 can include safety glasses or safety goggles, as discussed below. In particular, the eye protector 105 can be configured to protect the user's eyes from flying debris. For example, many environments include dust or other debris. This debris can often move in an unexpected direction and, without adequate protection, the user's eyes can be severely damaged by this debris.

Additionally or alternatively, the eye protector 105 can protect the user's eyes from overly bright light. For example, when welding the amount of light near the user's eyes can be quite high. The eye protector 105 can include tinting or other protection which restricts the amount of light passing through the eye protector 105 and reaching the user's eyes.

FIGS. 1A, 1B and 1C also show that the eye protector 105 can include a frame 110. In at least one implementation, the frame 110 can be configured to position the eye protector 105 in the correct position while in use. For example, the frame 110 can be configured to fit snugly on the user's head in order to position the eye protector 105 for correct use. Additionally or alternatively, the frame 110 can include one or more nose pads. The nose pads can be configured to allow the frame 110 to rest on the user's nose, such that the eye protector 105 is positioned correctly while in use. Additionally or alternatively, the frame 110 can include ear pieces. The ear pieces can be configured to hold the frame 110 in place relative the user's ears. For example, the frame 110 can include a curved portion which is configured to be placed behind the user's ears. This can prevent the frame 110 from falling off when the user leans forward.

In at least one implementation, the frame 110 can include a wrap-around frame. A wrap-around frame includes any frame 110 which is configured to wrap around the user's eyes, such that debris moving from the side, rather than from in front of the wearer, is likewise prevented from striking the user's eyes. Additionally or alternatively, the frame 110 can include a side protector. In at least one implementation, a side protector is connected to the frame and configured to protect the user's eyes from debris which come from the side.

FIGS. 1A, 1B and 1C further show that the eye protector 105 can include one or more lenses 115. In at least one implementation, the one or more lenses 115 can be configured to withstand the impact of flying debris. For example, the one or more lenses 115 can be sufficiently hard that flying debris strikes the one or more lenses 115 and is prevented from entering the user's eyes.

In at least one implementation, the one or more lenses 115 can be shatterproof. As used in the specification and claims, the term shatterproof shall mean that the material resists shattering or that the material produces rounded grains rather than jagged grains when broken, unless otherwise specified. Shatterproof lenses 115 can prevent the flying debris from shattering the one or more lenses 115 and creating additional debris that can damage the user's eyes. For example, if the one or more lenses 115 are made of standard glass, a forceful impact can shatter the glass, producing a number of sharp grains that may be moving in the direction of the user's eyes. In contrast, in the one or more lenses 115 are made of shatterproof material, a forceful impact will either crack, but not shatter, the one or more lenses or any debris produced will be larger and lack sharp edges which could damage the user's eyes.

In at least one implementation, the one or more lenses 115 can be made of any material which is configured to protect the user's eyes. For example the one or more lenses 115 can be made of glass, plastic, polycarbonate, trivex, polyurethane or any other suitable material. The material can be configured to allow the material to meet safety specifications. For example, the material can be configured to meet the safety glasses standard provided in publication number z87.1 by the American National Standards Institute (ANSI).

Additionally or alternatively, the one or more lenses 115 can be thick enough to protect the user's eyes in against flying debris. The one or more lenses 115 can be between 0.75 millimeters and 1.25 millimeters thick. For example, the one or more lenses 115 can be approximately 1.0 millimeters thick. As used in the specification and the claims, the term approximately shall mean that the value is within 10% of the stated value, unless otherwise specified.

In at least one implementation, the one or more lenses 115 can be made of a transparent, or substantially transparent, material. As used in the specification, the term transparent shall mean that the material is capable of transmitting light so that objects or images can be seen as if there were no intervening material, unless otherwise specified. I.e., the user is able to see through the material without a substantial decrease in the amount of light which passes through the one or more lenses 115.

Additionally or alternatively, the one or more lenses 115 can prevent a portion of the light from passing through the one or more lenses 115. For example, the one or more lenses 115 can be tinted. In at last one implementation, tinting the one or more lenses 115 can include placing a film over the one or more lenses 115 which reduces the amount of light which is transmitted through the one or more lenses 115. Additionally or alternatively, the material itself can reduce the amount of light transmitted through the one or more lenses.

FIGS. 1A, 1B and 1C also show that the system 100 can include a hearing protector 120. In at least one implementation, the hearing protector 120 can include any device designed to prevent noise-induced hearing loss. Noise-induced hearing loss can include any damage to a user's ears including physical damage and a reduction in the sensitivity of the user's hearing.

FIGS. 1A, 1B and 1C further show that the hearing protector 120 can include a first earplug 125a and a second earplug 125b (collectively “earplugs 125”). In at least one implementation an earplug 125 can include a device that is meant to be inserted in the ear canal to protect the wearer's ears from loud noises or the intrusion of water, foreign bodies, dust or excessive wind. I.e., an earplug 125 can include any device that is inserted in the ear canal in order to protect the user's ears. Additionally or alternatively, the hearing protector 120 can include ear muffs, as discussed below.

In at least one implementation, the earplugs 125 can include any material intended to protect the user's hearing. For example, the earplugs 125 can include foam, silicone, flanged earplugs or any other material. In particular, the earplugs 125 can include any material which can be inserted into the ear canal without damaging the user's ears.

FIGS. 1A, 1B and 1C also show that the system 100 can include a first connector 130a and a second connector 130b (collectively “connectors 130”). In at least one implementation, the first connector 130a and the second connector 130b are configured to attach the hearing protector 120 to the frame 110. The connectors 130 can include any material which allows the hearing protector 120 to be connected to the frame 110. For example, the connectors 130 can include a nylon cord attached to the frame 110 and the hearing protector 120.

In at least one implementation, the connectors 130 can be configured to releasably attach to the frame 110, the hearing protector 120 or both. I.e., the connectors can be attached and detached from the frame 110 and/or the hearing protector 120 by the user as desired. For example, the connector can include a snap, a hook and loop fastener, a tie or any other attachment mechanism. The user may wish to detach the hearing protector 120 from the frames for a number of reasons. E.g., the user may desire to wash the hearing protector 120 the frame 110 or both. Alternatively, the user may desire to use only the eye protector 105 or the hearing protector 120 within a certain environment.

In at least on implantation, the connectors 130 can ensure that the hearing protector 120 is near when needed. In particular, as the user enters an area where loud noise is likely or as machinery or loud equipment is turned on, the user need not search for the hearing protector 120. This results in quicker use of the hearing protector 120 and a consequent increase in hearing protection.

FIGS. 1A, 1B and 1C further show that the system 100 can include a pencil holder 135. In at least one implementation, the user can safely place a pencil 140, pen, marker, or other writing instrument in the pencil holder 135. In particular, often users place a pencil 140 over their ear. It can move and poke the user or slip where it can become a hazard on the floor. The pencil holder 135 can ensure that the user can quickly place the pencil 140 in a safe manner.

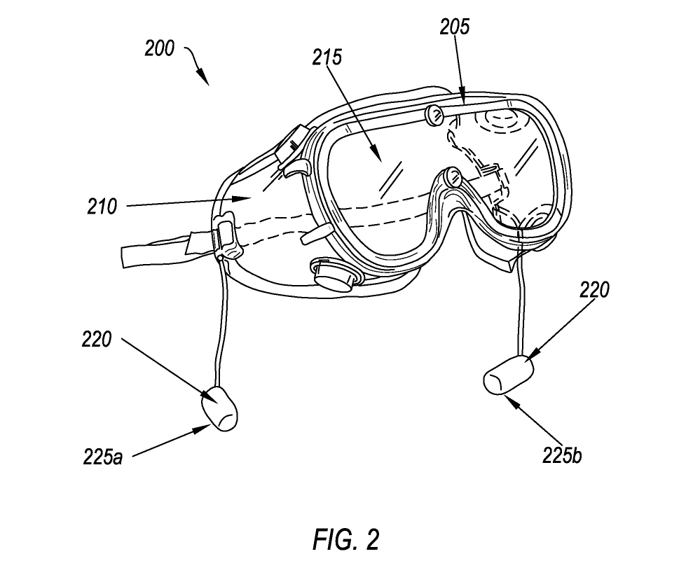

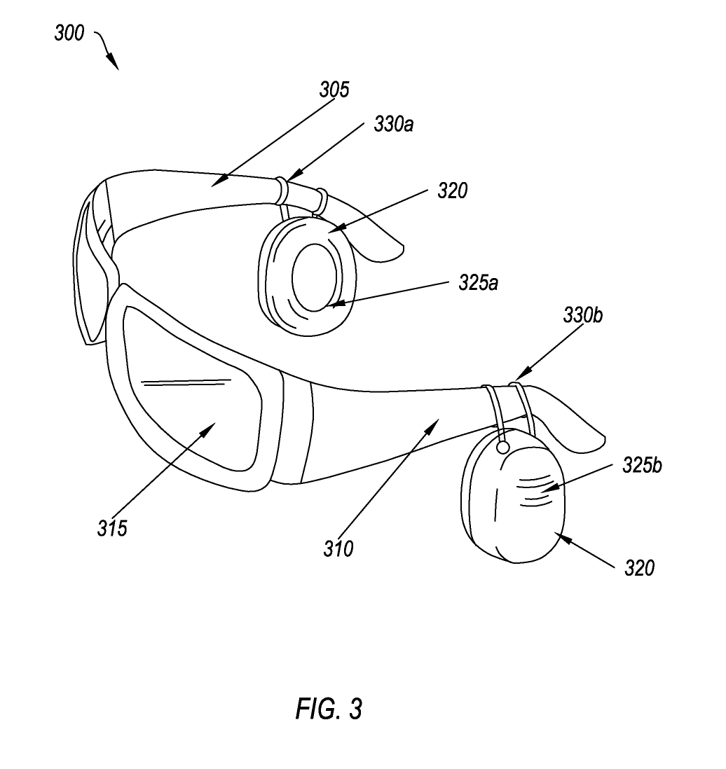

FIG. 2 illustrates an alternative system 200 of increasing worker safety. In at least one implementation, the system 200 can be used to protect a worker in a dangerous environment. For example, in construction zones, manufacturing areas or anywhere else that the user is likely to be exposed to loud noises or moving debris the system 200 can protect the user's eyes and hearing.

FIG. 2 shows what the system 200 can include an eye protector 205. In at least one implementation, the eye protector 205 can serve to protect the user's eyes. For example, the eye protector 205 can include safety goggles. In particular, the eye protector 205 can be configured to protect the user's eyes from flying debris. For example, many environments include dust or other debris. This debris can often move in an unexpected direction and, without adequate protection, the user's eyes can be severely damaged by this debris.

Additionally or alternatively, the eye protector 205 can protect the user's eyes from overly bright light. For example, when welding the amount of light near the user's eyes can be quite high. The eye protector 205 can include tinting or other protection which restricts the amount of light passing through the eye protector 205 and reaching the user's eyes.

FIG. 2 also shows that the eye protector 205 can include a casing 210. In at least one implementation, the casing 210 can be configured to fit snugly around the user's face. I.e., the casing 210 can make continuous contact with the face of the user in order to protect the user's eyes. In particular, chemical splashes or other hazards in which the user's eyes are in particular danger from even a small amount of debris entering the eyes can be protected against by eliminating all direct paths from outside the casing 210 to inside the casing 210. For example, the eye protector 205 can include goggles that enclose the area surrounding the eye in order to prevent particulates, water or chemicals from striking the user's eyes.

In at least one implementation, the casing 210 can include any material that is configured to protect the user's eyes. For example, the casing 210 can include rubber, plastic, metal or any other suitable material. In particular, the casing 210 can be constructed of different materials to prevent against hazards found in the user's environment. For example, the casing 210 can be made of transparent flexible material when the user is working with chemicals in order to prevent chemicals from getting near the user's eyes without restricting vision. In contrast, the casing 210 can be made of hard plastic when the user is working in a machine shop or other environment with a high amount of particulate matter.

FIG. 2 further shows that the eye protector 205 can include one or more lenses 215. In at least one implementation, the one or more lenses 215 can be configured to withstand the impact of flying debris. For example, the one or more lenses can be sufficiently hard that flying debris strikes the one or more lenses 215 and is prevented from entering the user's eyes.

In at least one implementation, the one or more lenses 215 can be shatterproof. Shatterproof lenses 215 can prevent the flying debris from shattering the one or more lenses 215 and creating additional debris that can damage the user's eyes. For example, if the one or more lenses 215 are made of standard glass, a forceful impact can shatter the glass, producing a number of sharp grains that may be moving in the direction of the user's eyes. In contrast, in the one or more lenses 215 are made of shatterproof material, a forceful impact will either crack, but not shatter, the one or more lenses or any debris produced will be larger and lack sharp edges which could damage the user's eyes.

(P20) In at least one implementation, the one or more lenses 215 can be made of any material which is configured to protect the user's eyes. For example the one or more lenses 215 can be made of glass, plastic, polycarbonate, trivex, polyurethane or any other suitable material. The material can be configured to allow the material to meet safety specifications. For example, the material can be configured to meet the safety glasses standard provided in publication number z87.1 by the ANSI.

Additionally or alternatively, the one or more lenses 215 can be thick enough to protect the user's eyes in against flying debris. For example, the one or more lenses 215 can be between 0.75 millimeters and 1.25 millimeters thick. E.g., the one or more lenses 215 can be approximately 1.0 millimeters thick.

In at least one implementation, the one or more lenses 215 can be made of a transparent, or substantially transparent, material. I.e., the user is able to see through the material without a substantial decrease in the amount of light which passes through the one or more lenses 215.

Additionally or alternatively, the one or more lenses 215 can prevent a portion of the light from passing through the one or more lenses 215. For example, the one or more lenses 215 can be tinted. In at last one implementation, tinting the one or more lenses 215 can include placing a film over the one or more lenses 215 which reduces the amount of light which is transmitted through the one or more lenses 215. Additionally or alternatively, the material itself can reduce the amount of light transmitted through the one or more lenses.

FIG. 2 also shows that the system 200 can include a hearing protector 220. In at least one implementation, the hearing protector 220 can include any device designed to prevent noise-induced hearing loss. Noise-induced hearing loss can include any damage to a user's ears including physical damage and a reduction in the sensitivity of the user's hearing.

FIG. 2 further shows that the hearing protector 220 can include a first earplug 225a and a second earplug 225b (collectively “earplugs 225”). In at least one implementation an earplug 225 can include a device that is meant to be inserted in the ear canal to protect the wearer's ears from loud noises or the intrusion of water, foreign bodies, dust or excessive wind. I.e., an earplug 225 can include any device that is inserted in the ear canal in order to protect the user's ears. Additionally or alternatively, the hearing protector 220 can include ear muffs, as discussed below.

In at least one implementation, the earplugs 225 can include any material intended to protect the user's hearing. For example, the earplugs 225 can include foam, silicone, flanged earplugs or any other material. In particular, the earplugs 225 can include any material which can be inserted into the ear canal without damaging the user's ears.

FIG. 2 also shows that the system 200 can include a first connector 230a and a second connector 230b (collectively “connectors 230”). In at least one implementation, the first connector 230a and the second connector 230b are configured to attach the first hearing protector 220 to the casing 210. The connectors 230 can include any material which allows the hearing protector 220 to be connected to the casing 210. For example, the connectors 230 can include a nylon cord attached to the casing 210 and the hearing protector 220.

In at least one implementation, the connectors 230 can be configured to releasably attach to the casing 210, the hearing protector 220 or both. I.e., the connectors can be attached and detached from the casing 210 and/or the hearing protector 220 by the user as desired. For example, the connector can include a snap, a hook and loop fastener, a tie or any other attachment mechanism. The user may wish to detach the hearing protector 220 from the frames for a number of reasons. E.g., the user may desire to wash the hearing protector 220 the casing 210 or both. Alternatively, the user may desire to use only the eye protector 205 or the hearing protector 220 within a certain environment.

In at least on implantation, the connectors 230 can ensure that the hearing protector 220 is near when needed. In particular, as the user enters an area where loud noise is likely or as machinery or loud equipment is turned on, the user need not search for the hearing protector 120. This results in quicker use of the hearing protector 120 and a consequent increase in hearing protection.

FIG. 3 illustrates an alternative system 300 of increasing worker safety. In at least one implementation, the system 300 can be used to protect a worker in a dangerous environment. For example, in construction zones, manufacturing areas or anywhere else that the user is likely to be exposed to loud noises or moving debris the system 300 can protect the user's eyes and hearing.

FIG. 3 shows what the system 300 can include an eye protector 305. In at least one implementation, the eye protector 305 can serve to protect the user's eyes. For example, the eye protector 305 can include safety glasses or safety goggles. In particular, the eye protector 305 can be configured to protect the user's eyes from flying debris. For example, many environments include dust or other debris. This debris can often move in an unexpected direction and, without adequate protection, the user's eyes can be severely damaged by this debris.

Additionally or alternatively, the eye protector 305 can protect the user's eyes from overly bright light. For example, when welding the amount of light near the user's eyes can be quite high. The eye protector 305 can include tinting or other protection which restricts the amount of light passing through the eye protector 305 and reaching the user's eyes.

FIG. 3 also shows that the eye protector 305 can include a frame 310. In at least one implementation, the frame 310 can be configured to position the eye protector 305 in the correct position while in use. For example, the frame 310 can be configured to fit snugly on the user's head in order to position the eye protector 305 for correct use. Additionally or alternatively, the frame 310 can include one or more nose pads. The nose pads can be configured to allow the frame 310 to rest on the user's nose, such that the eye protector 305 is positioned correctly while in use. Additionally or alternatively, the frame 310 can include ear pieces. The ear pieces can be configured to hold the frame 310 in place relative the user's ears. For example, the frame 310 can include a curved portion which is configured to be placed behind the user's ears. This can prevent the frame 310 from falling off when the user leans forward.

In at least one implementation, the frame 310 can include a wrap-around frame. A wrap-around frame includes any frame 310 which is configured to wrap around the user's eyes, such that debris moving from the side, rather than from in front of the wearer, is likewise prevented from striking the user's eyes. Additionally or alternatively, the frame 310 can include a side protector. In at least one implementation, a side protector is connected to the frame and configured to protect the user's eyes from debris which come from the side.

FIG. 3 further shows that the eye protector 305 can include one or more lenses 315. In at least one implementation, the one or more lenses 315 can be configured to withstand the impact of flying debris. For example, the one or more lenses 315 can be sufficiently hard that flying debris strikes the one or more lenses 315 and is prevented from entering the user's eyes.

In at least one implementation, the one or more lenses 315 can be shatterproof. Shatterproof lenses 315 can prevent the flying debris from shattering the one or more lenses 315 and creating additional debris that can damage the user's eyes. For example, if the one or more lenses 315 are made of standard glass, a forceful impact can shatter the glass, producing a number of sharp grains that may be moving in the direction of the user's eyes. In contrast, in the one or more lenses 315 are made of shatterproof material, a forceful impact will either crack, but not shatter, the one or more lenses or any debris produced will be larger and lack sharp edges which could damage the user's eyes.

In at least one implementation, the one or more lenses 315 can be made of any material which is configured to protect the user's eyes. For example the one or more lenses 315 can be made of glass, plastic, polycarbonate, trivex, polyurethane or any other suitable material. The material can be configured to allow the material to meet safety specifications. For example, the material can be configured to meet the safety glasses standard provided in publication number z87.1 by the ANSI.

Additionally or alternatively, the one or more lenses 315 can be thick enough to protect the user's eyes in against flying debris. For example, the one or more lenses 315 can be between 0.75 millimeters and 1.25 millimeters thick. E.g., the one or more lenses 315 can be approximately 1.0 millimeters thick.

In at least one implementation, the one or more lenses 315 can be made of a transparent, or substantially transparent, material. As used in the specification, the term transparent shall mean that the material is capable of transmitting light so that objects or images can be seen as if there were no intervening material, unless otherwise specified. I.e., the user is able to see through the material without a substantial decrease in the amount of light which passes through the one or more lenses 315.

Additionally or alternatively, the one or more lenses 315 can prevent a portion of the light from passing through the one or more lenses 315. For example, the one or more lenses 315 can be tinted. In at last one implementation, tinting the one or more lenses 315 can include placing a film over the one or more lenses 315 which reduces the amount of light which is transmitted through the one or more lenses 315. Additionally or alternatively, the material itself can reduce the amount of light transmitted through the one or more lenses.

FIG. 3 also shows that the system 300 can include a hearing protector 320. In at least one implementation, the hearing protector 320 can include any device designed to prevent noise-induced hearing loss. Noise-induced hearing loss can include any damage to a user's ears including physical damage and a reduction in the sensitivity of the user's hearing.

FIG. 3 further shows that the hearing protector 320 can include a first earmuff 325a and a second earmuff 325b (collectively “earmuffs 325”). In at least one implementation, the earmuffs 125 are objects designed to cover a user's ears in order to provide hearing protection. For example, the earmuffs 125 can include cups lined with sound-deadening material which are placed over the user's external ear. In particular, the earmuffs 125 can include acoustic foam or other material that absorbs sound waves by increasing air resistance, thus reducing the amplitude of the sound waves.

In at least one implementation, the earmuffs 125 can include active sound protection. I.e., the earmuffs 125 can include a microphone mounted in the headset. The microphone can receive ambient sounds and transmit them through a dynamic range compression circuit to earphones inside the earmuffs 125. By virtue of the dynamic compression, the active sound protection can be adjusted to allow the wearer to hear sounds at ordinary volumes normally, while attenuating louder sounds.

In at least one implementation, the earmuffs 125 can include headphones. The headphones can transmit music, communication or other audio sources to the user. I.e., the headphones can protect the user's ears from ambient noise while simultaneously transmitting to the user needed communication at ordinary volumes.

In at least one implementation, the earmuffs 325 can include a headband. The headband can be configured to fit over the top of the user's head or behind the user's head in order to place the cups as desired by the user. For example, the headband can include an adjustment mechanism that allows the user to adjust the length of the headband. Thus, when the headband rests on the user's head, the cups of the earmuff 125 rest over the user's ears. The headband can be made of any material suitable for supporting the cups of the earmuff 125 over the user's ears. For example, they can include thermoplastic, metal or any other suitable material.

FIG. 3 also shows that the system 300 can include a first connector 330a and a second connector 330b (collectively “connectors 330). In at least one implementation, the first connector 330a and the second connector 330b are configured to attach the haring protector 320 to the frame 310. The connectors 330 can include any material which allows the hearing protector 320 to be connected to the frame 310. For example, the connectors 330 can include a clip attached to the frame 310 and the hearing protector 320.

In at least one implementation, the connectors 330 can be configured to releasably attach to the frame 310, the hearing protector 320 or both. I.e., the connectors can be attached and detached from the frame 310 and/or the hearing protector 320 by the user as desired. For example, the connector can include a snap, a hook and loop fastener, a tie or any other attachment mechanism. The user may wish to detach the hearing protector 320 from the frames for a number of reasons. E.g., the user may desire to wash the hearing protector 320 the frame 310 or both. Alternatively, the user may desire to use only the eye protector 305 or the hearing protector 320 within a certain environment.

In at least on implantation, the connectors 330 can ensure that the hearing protector 320 is near when needed. In particular, as the user enters an area where loud noise is likely or as machinery or loud equipment is turned on, the user need not search for the hearing protector 320. This results in quicker use of the hearing protector 320 and a consequent increase in hearing protection.

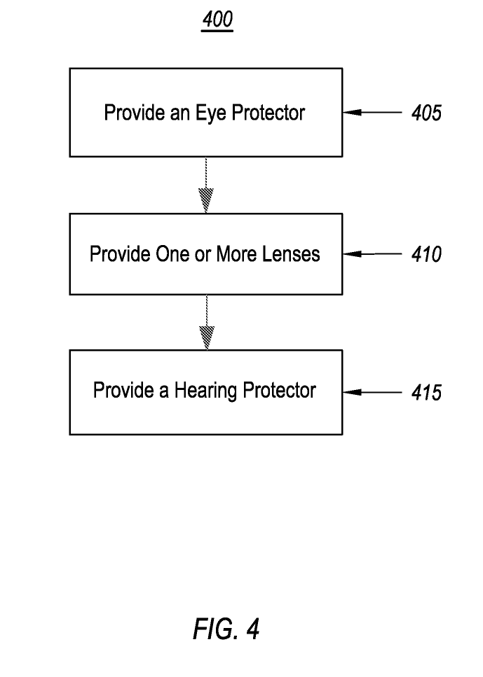

FIG. 4 is a flow chart illustrating a method 400 of manufacturing a system for increasing worker safety. One of skill in the art will appreciate that the method 400 can be used to produce the system 100 of FIG. 1, the system 200 of FIG. 2 or the system 300 of FIG. 3; however, the method 400 can be used to produce a system other than the system 100 of FIG. 1, the system 200 of FIG. 2 or the system 300 of FIG. 3. In at least one implementation, the system can be used to protect a worker in a dangerous environment. For example, in construction zones, manufacturing areas or anywhere else that the user is likely to be exposed to loud noises or moving debris the system can protect the user's eyes and hearing.

FIG. 4 shows that the method 400 can include providing an eye protector 405. In at least one implementation, the eye protector can serve to protect the user's eyes. For example, the eye protector can include safety glasses or safety goggles, as discussed below. In particular, the eye protector can be configured to protect the user's eyes from flying debris. For example, many environments include dust or other debris. This debris can often move in an unexpected direction and, without adequate protection, the user's eyes can be severely damaged by this debris.

Additionally or alternatively, the eye protector can protect the user's eyes from overly bright light. For example, when welding the amount of light near the user's eyes can be quite high. The eye protector can include tinting or other protection which restricts the amount of light passing through the eye protector and reaching the user's eyes.

In at least one implementation, the eye protector can include a frame. The frame can be configured to position the eye protector in the correct position while in use. For example, the frame can be configured to fit snugly on the user's head in order to position the eye protector for correct use. Additionally or alternatively, the frame can include one or more nose pads. The nose pads can be configured to allow the frame to rest on the user's nose, such that the eye protector is positioned correctly while in use. Additionally or alternatively, the frame can include ear pieces. The ear pieces can be configured to hold the frame in place relative the user's ears. For example, the frame can include a curved portion which is configured to be placed behind the user's ears. This can prevent the frame from falling off when the user leans forward.

In at least one implementation, the frame can include a wrap-around frame. A wrap-around frame includes any frame which is configured to wrap around the user's eyes, such that debris moving from the side, rather than from in front of the wearer, is likewise prevented from striking the user's eyes. Additionally or alternatively, the frame can include a side protector. In at least one implementation, a side protector is connected to the frame and configured to protect the user's eyes from debris which come from the side.

Additionally or alternatively, the eye protector can include a casing. In at least one implementation, the casing can be configured to fit snugly around the user's face. I.e., the casing can make continuous contact with the face of the user in order to protect the user's eyes. In particular, chemical splashes or other hazards in which the user's eyes are in particular danger from even a small amount of debris entering the eyes can be protected against by eliminating all direct paths from outside the casing to inside the casing. For example, the eye protector can include goggles that enclose the area surrounding the eye in order to prevent particulates, water or chemicals from striking the user's eyes.

In at least one implementation, the casing can include any material that is configured to protect the user's eyes. For example, the casing can include rubber, plastic, metal or any other suitable material. In particular, the casing can be constructed of different materials to prevent against hazards found in the user's environment. For example, the casing can be made of transparent flexible material when the user is working with chemicals in order to prevent chemicals from getting near the user's eyes without restricting vision. In contrast, the casing can be made of hard plastic when the user is working in a machine shop or other environment with a high amount of particulate matter.

FIG. 4 also shows that the method 400 can include providing one ore more lenses 410. In at least one implementation, the one or more lenses can be configured to withstand the impact of flying debris. For example, the one or more lenses can be sufficiently hard that flying debris strikes the one or more lenses and is prevented from entering the user's eyes.

In at least one implementation, the one or more lenses can be shatterproof. Shatterproof lenses can prevent the flying debris from shattering the one or more lenses and creating additional debris that can damage the user's eyes. For example, if the one or more lenses are made of standard glass, a forceful impact can shatter the glass, producing a number of sharp grains that may be moving in the direction of the user's eyes. In contrast, in the one or more lenses are made of shatterproof material, a forceful impact will either crack, but not shatter, the one or more lenses or any debris produced will be larger and lack sharp edges which could damage the user's eyes.

In at least one implementation, the one or more lenses can be made of any material which is configured to protect the user's eyes. For example the one or more lenses can be made of glass, plastic, polycarbonate, trivex, polyurethane or any other suitable material. The material can be configured to allow the material to meet safety specifications. For example, the material can be configured to meet the safety glasses standard provided in publication number z87.1 by the ANSI.

Additionally or alternatively, the one or more lenses can be thick enough to protect the user's eyes in against flying debris. The one or more lenses can be between 0.75 millimeters and 1.25 millimeters thick. For example, the one or more lenses can be approximately 1.0 millimeters thick. As used in the specification and the claims, the term approximately shall mean that the value is within 10% of the stated value, unless otherwise specified.

In at least one implementation, the one or more lenses can be made of a transparent, or substantially transparent, material. I.e., the user is able to see through the material without a substantial decrease in the amount of light which passes through the one or more lenses.

Additionally or alternatively, the one or more lenses can prevent a portion of the light from passing through the one or more lenses. For example, the one or more lenses can be tinted. In at last one implementation, tinting the one or more lenses can include placing a film over the one or more lenses which reduces the amount of light which is transmitted through the one or more lenses. Additionally or alternatively, the material itself can reduce the amount of light transmitted through the one or more lenses.

FIG. 4 also shows that the method 400 can include providing a hearing protector 415. In at least one implementation, the hearing protector can include any device designed to prevent noise-induced hearing loss. Noise-induced hearing loss can include any damage to a user's ears including physical damage and a reduction in the sensitivity of the user's hearing.

In at least one implementation, the hearing protector can include a first earplug and a second earplug. The earplug can include a device that is meant to be inserted in the ear canal to protect the wearer's ears from loud noises or the intrusion of water, foreign bodies, dust or excessive wind. I.e., an earplug can include any device that is inserted in the ear canal in order to protect the user's ears. Additionally or alternatively, the hearing protector can include ear muffs, as discussed below.

In at least one implementation, the earplugs can include any material intended to protect the user's hearing. For example, the earplugs can include foam, silicone, flanged earplugs or any other material. In particular, the earplugs can include any material which can be inserted into the ear canal without damaging the user's ears.

Additionally or alternatively, the hearing protector can include a first earmuff and a second earmuff. In at least one implementation, the earmuffs are objects designed to cover a user's ears in order to provide hearing protection. For example, the earmuffs can include cups lined with sound-deadening material which are placed over the user's external ear. In particular, the earmuffs can include acoustic foam or other material that absorbs sound waves by increasing air resistance, thus reducing the amplitude of the sound waves.

In at least one implementation, the earmuffs can include active sound protection. I.e., the earmuffs can include a microphone mounted in the headset. The microphone can receive ambient sounds and transmit them through a dynamic range compression circuit to earphones inside the ear muffs. By virtue of the dynamic compression, the active sound protection can be adjusted to allow the wearer to hear sounds at ordinary volumes normally, while attenuating louder sounds.

In at least one implementation, the earmuffs can include headphones. The headphones can transmit music, communication or other audio sources to the user. I.e., the headphones can protect the user's ears from ambient noise while simultaneously transmitting to the user needed communication at ordinary volumes.

In at least one implementation, the earmuffs can include a headband. The headband can be configured to fit over the top of the user's head or behind the user's head in order to place the cups as desired by the user. For example, the headband can include an adjustment mechanism that allows the user to adjust the length of the headband. Thus, when the headband rests on the user's head, the cups of the earmuff rest over the user's ears. The headband can be made of any material suitable for supporting the cups of the earmuff over the user's ears. For example, they can include thermoplastic, metal or any other suitable material.

In at least one implementation, the system can include a first connector and a second connector. The first connector and the second connector are configured to attach the hearing protector to the frame. The connectors can include any material which allows the hearing protector to be connected to the frame. For example, the connectors can include a nylon cord attached to the frame and the hearing protector.

In at least one implementation, the connectors can be configured to releasably attach to the frame, the hearing protector or both. I.e., the connectors can be attached and detached from the frame and/or the hearing protector by the user as desired. For example, the connector can include a snap, a hook and loop fastener, a tie or any other attachment mechanism. The user may wish to detach the hearing protector from the frames for a number of reasons. E.g., the user may desire to wash the hearing protector the frame or both. Alternatively, the user may desire to use only the eye protector or the hearing protector within a certain environment.

In at least on implantation, the connectors can ensure that the hearing protector is near when needed. In particular, as the user enters an area where loud noise is likely or as machinery or loud equipment is turned on, the user need not search for the hearing protector. This results in quicker use of the hearing protector and a consequent increase in hearing protection.

In at least one implementation, the system can include a pencil holder. The user can safely place a pencil, pen, marker, or other writing instrument in the pencil holder. In particular, often users place a pencil over their ear. It can move and poke the user or slip where it can become a hazard on the floor. The pencil holder can ensure that the user can quickly place the pencil in a safe manner.

One of skill in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system for protecting a worker in a dangerous environment, the system comprising:
an eye protector, wherein the eye protector includes:
one or more lenses, wherein the one or more lenses are:
configured to be positioned in front of the user's eyes;
configured to protect the user's eyes; and
a hearing protector, wherein the hearing protector is configured to protect the user's hearing from damage.

2. The system of claim 1, wherein the eye protector includes a frame, wherein the frame is configured to locate the eye protector in front of the user's eyes.

3. The system of claim 2 further comprising a connector, wherein the connector is configured to attach the hearing protector to the frame.

4. The system of claim 1, wherein the one or more lenses are shatter resistant.

5. The system of claim 1, wherein the one or more lenses include plastic.

6. The system of claim 1, wherein the one or more lenses are:
transparent; or
substantially transparent.

7. The system of claim 1, wherein the one or more lenses are tinted.

8. The system of claim 1, wherein the hearing protector includes earplugs.

9. The system of claim 1, wherein the system further includes a pencil holder.

10. A system for protecting a worker in a dangerous environment, the system comprising:
an eye protector, wherein the eye protector includes:
a frame, wherein the frame is configured to locate the eye protector in front of the user's eyes;
one or more lenses, wherein the one or more lenses are:
held within the frame in front of the user's eyes;
configured to protect the user's eyes; and
a hearing protector, wherein the hearing protector includes:
a left ear protector; and
a right ear protector;
wherein the hearing protector is configured to protect the user's hearing from damage.

11. The system of claim 10, wherein the eye protector includes one or more side protectors, wherein the one or more side protectors are connected to the frame and configured to protect the user's eyes from debris which come from the side.

12. The system of claim 10, wherein the one or more lenses:
are configured to meet the American National Standards Institute requirements for safety glasses.

13. The system of claim 10, wherein the minimum thickness of the one or more lenses is between 0.75 millimeters and 1.25 millimeters.

14. The system of claim 13, wherein the minimum thickness of the one or more lenses is approximately 1.0 millimeters

15. The system of claim 10, wherein the one or more lenses include one or:
polycarbonate; or
trivex.

16. A method of manufacturing a system for protecting a worker in a dangerous environment, the method comprising:
providing an eye protector, wherein the eye protector includes:
one or more lenses, wherein the one or more lenses are:
configured to be positioned in front of the user's eyes;
configured to protect the user's eyes; and
providing a hearing protector, wherein the hearing protector is configured to protect the user's hearing from damage.

17. The system of claim 16, wherein the hearing protector includes ear muffs.

18. The system of claim 17, wherein the ear muffs include a pad on each end, wherein the pad is configured to cover the external ears.

19. The system of claim 18, wherein the ear muffs include a head-band, wherein the head-band is configured to fit over the top of the user's head.

20. The system of claim 19, wherein the head-band includes one of:
thermoplastic; or
metal.
